(19) United States
(12) Patent Application Publication
(10) Pub. No.: US 2015/0032021 A1
(43) Pub. Date: Jan. 29, 2015

(54) SAFETY MONITORING SYSTEM AND FATIGUE MONITORING APPARATUS AND FATIGUE DETECTING HELMET THEREOF

(71) Applicant: AmTRAN Technology Co., Ltd., New Taipei City (TW)

(72) Inventors: Jian-Lin CHEN, New Taipei City (TW); Hui-An KO, Taipei City (TW); Yang-Hsu LAI, New Taipei City (TW); Huai-Shan CHEN, New Taipei City (TW); Shao-Chin CHANG, Taichung City (TW); Hsu-Hsuan WU, Taipei City (TW)

(73) Assignee: AmTRAN Technology Co., Ltd., New Taipei City (TW)

(21) Appl. No.: 14/086,955

(22) Filed: Nov. 21, 2013

(30) Foreign Application Priority Data
Jul. 23, 2013 (TW) 102126308

(51) Int. Cl. A61B 5/00; A61B 5/0478

(52) U.S. Cl. CPC A61B 5/7275; A61B 5/0478; A61B 5/6814; A61B 5/746
USPC 600/544

(57) ABSTRACT

A fatigue monitoring apparatus includes a fatigue detecting helmet. The fatigue detecting helmet includes a cap, a first electrode, a second electrode, a fatigue computation device and a power supply. The first electrode is disposed in the cap to contact a head, thereby obtaining a first physiological information. The second electrode is positioned out of the cap to contact the head, thereby obtaining a second physiological information. The fatigue computation device and the power supply are disposed on the cap. The fatigue computation device includes a brainwave computation module and a fatigue judgment module. The brainwave computation module is electrically connected to the first and second electrodes for obtaining a brainwave information based on the first and second physiological information. The fatigue judgment module is electrically connected to the brainwave computation module for obtaining a fatigue information based on the brainwave information.

### RELATED APPLICATIONS

This application claims priority to Taiwanese Application Serial Number 102126308, filed Jul. 23, 2013, which is herein incorporated by reference.

### BACKGROUND

1. Technical Field Embodiments of the present invention relate to a monitoring system.

More particularly, embodiments of the present invention relate to a safety monitoring system and the fatigue monitoring apparatus capable of monitoring the fatigue of the user.

2. Description of Related Art

Industrial safety is always one of the main issues in a construction site. Many accidents in construction sites result from the human negligence of workers, which is usually caused by fatigue. For example, the worker on duty may lose attention and suffer from fatigue because of lacking for reasonable rest time or constantly working for a long time, which could be more than 8 hours. When a worker loses his attention and is still engaged in a highly risky work, an accident may occur and endangering the worker's life.

Therefore, how to monitor the fatigue level of the worker on duty is a key issue to prevent the accidents.

### SUMMARY

A summary of various embodiments according to the present invention is disclosed below. It should be understood that these aspects are presented merely to provide the reader with a brief summary of these certain embodiments and that these aspects are not intended to limit the scope of this disclosure.

A safety monitoring system is provided, which can effectively monitor the fatigue of the worker by a helmet.

In accordance with one embodiment of the present invention, a fatigue detecting helmet includes a cap, at least one first electrode, at least one second electrode, a fatigue computation device and a power supply. The cap has a concave inner surface. The first electrode is disposed on the concave inner surface of the cap to be in contact with one position on a head of a wearer, thereby obtaining a first physiological information of the wearer. The second electrode is disposed out of the cap to be in contact with another position on the head of the wearer, thereby obtaining a second physiological information of the wearer. The fatigue computation device is disposed on one side of the cap, and includes a brainwave computation module and a fatigue determining module. The brainwave computation module is electrically connected to the first electrode and the second electrode for obtaining a brainwave physiological information based on the first physiological information and the second physiological information. The fatigue determining module is electrically connected to the brainwave computation module for obtaining a fatigue information of the wearer based on the brainwave information. The power supply is disposed on another side of the cap opposite to the fatigue computation device. The fatigue computation device is electrically connected to the power supply.

In accordance with another embodiment of the present invention, a fatigue monitoring apparatus is capable of being attached to a cap, and the fatigue monitoring apparatus includes at least one first electrode, at least one second electrode, a fatigue computation device and a power supply. The first electrode can be in contact with a first position of a head, thereby obtaining a first physiological information. The second electrode can be in contact with a second position of the head, thereby obtaining a second physiological information. The second position is farer from a brain of the head than the first position. The fatigue computation device is capable of being disposed on one side of the cap, and includes a brainwave computation module and a fatigue determining module. The brainwave computation module is electrically connected to the first electrode and the second electrode for obtaining a brainwave information based on the first physiological information and the second physiological information. The fatigue determining module is electrically connected to the brainwave computation module for obtaining a fatigue information based on the brainwave information. The power supply is capable of being disposed on another side of the cap opposite to the fatigue computation device. The fatigue computation device is electrically connected to the power supply.

In accordance with yet another embodiment of the present invention, a safety monitoring system includes a monitoring server and at least one fatigue monitoring apparatus. The monitoring server has a first wireless communication module. The fatigue monitoring apparatus is attached to a cap, and includes at least one first electrode, at least one second electrode, a fatigue computation device and a power supply. The first electrode and the second electrode are respectively in contact with different positions of a head for respectively obtaining a first physiological information and a second physiological information. The fatigue computation device obtains a brainwave information based on the difference between the first physiological information and the second physiological information, and obtains a fatigue information based on the brainwave information. The power supply is electrically connected to the fatigue computation device for providing power. The fatigue computation device and the power supply are disposed on opposite sides of the cap. The fatigue monitoring apparatus includes a second wireless communication module. The first wireless communication module is in wireless communication with the second wireless communication module. The monitoring server receives the fatigue information from the fatigue monitoring apparatus.

In the foregoing embodiments, the helmet can detect the brainwave of the human body by the first electrode and the second electrode, so that the fatigue information can be obtained. Hence, the user of the monitoring server can remotely monitor the fatigue of the worker. Further, because the fatigue computation device and the power supply are respectively disposed on opposite sides of the cap, the weight of the helmet can be balanced, so that when the worker puts the helmet on the head thereof, he (or she) will not feel uncomfortable due to the unbalanced weight.

It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the embodiment, with reference made to the accompanying drawings as follows:

FIG. 1 is a schematic view of the safety monitoring system in accordance with one embodiment of the present invention;

FIG. 2 is a perspective view of the helmet in FIG. 1;

FIG. 3 is a top view of the helmet in FIG. 2;

FIG. 4 is a cross-sectional view of the helmet taken along the direction of the width W in FIG. 3;

FIG. 5 is a functional diagram of the safety monitoring system in accordance with one embodiment of the present invention;

FIG. 6 is a perspective view of the fatigue computation device in accordance with one embodiment of the present invention;

FIG. 7 is an explosive view of the fatigue computation device;

FIG. 8 is a cross-sectional view of the fatigue computation device taken along A-A′ line in FIG. 6;

FIG. 9 is a cross-sectional view of the fatigue computation device taken along B-B′ line in FIG. 6;

FIG. 10 is a cross-sectional view of the fatigue computation device taken along C-C′ line in FIG. 6;

FIG. 11 is another perspective view of the fatigue computation device in accordance with one embodiment of the present invention; and

FIG. 12 is a cross-sectional view of the fatigue computation device taken along D-D′ line in FIG. 11.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

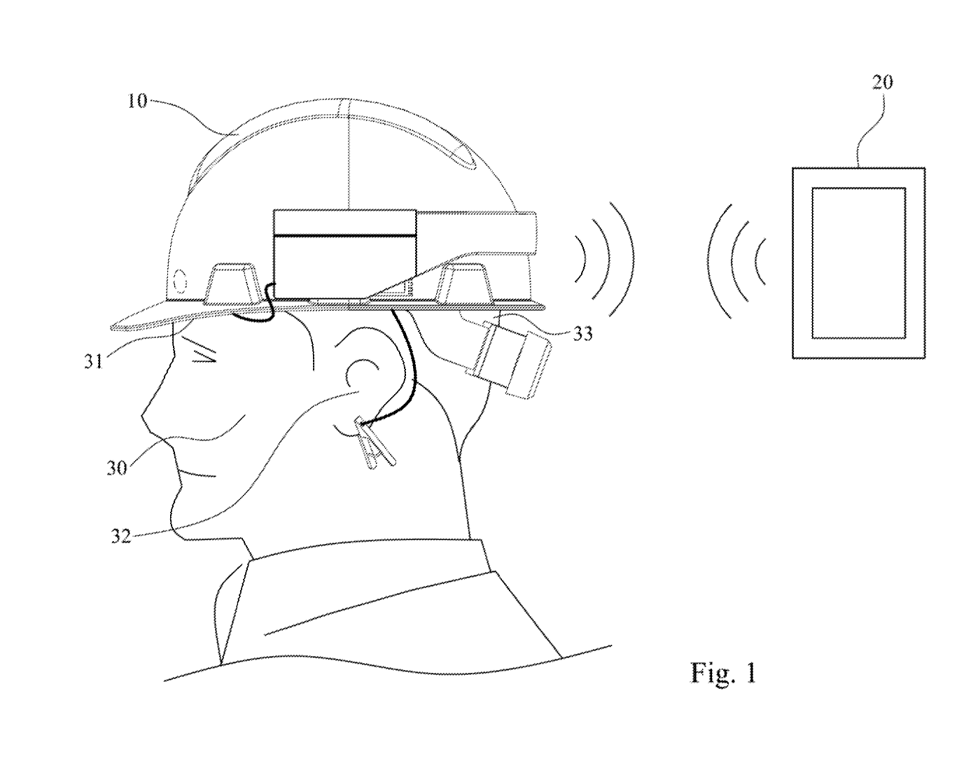

FIG. 1 is a schematic view of the safety monitoring system in accordance with one embodiment of the present invention. As shown in FIG. 1, the safety monitoring system includes at least one fatigue monitoring apparatus and a remote monitoring apparatus 20, such as a monitoring server. The fatigue monitoring apparatus can be attached to a helmet 10. The helmet 10 can be a typical safety helmet that can be put on a head 30 of a human. The helmet 10 with the fatigue monitoring apparatus can detect the physiological signals of the human including the brainwave, and obtain the fatigue information based on the brainwave. The remote monitoring apparatus 20 can receive the fatigue information from the helmet 10 via wireless communication. Accordingly, the user of the remote monitoring apparatus 20 can monitor the fatigue of every worker wearing the helmet 10. It is noted that the fatigue monitoring apparatus can be fastened on the helmet 10 without damaging the structure of the helmet 10.

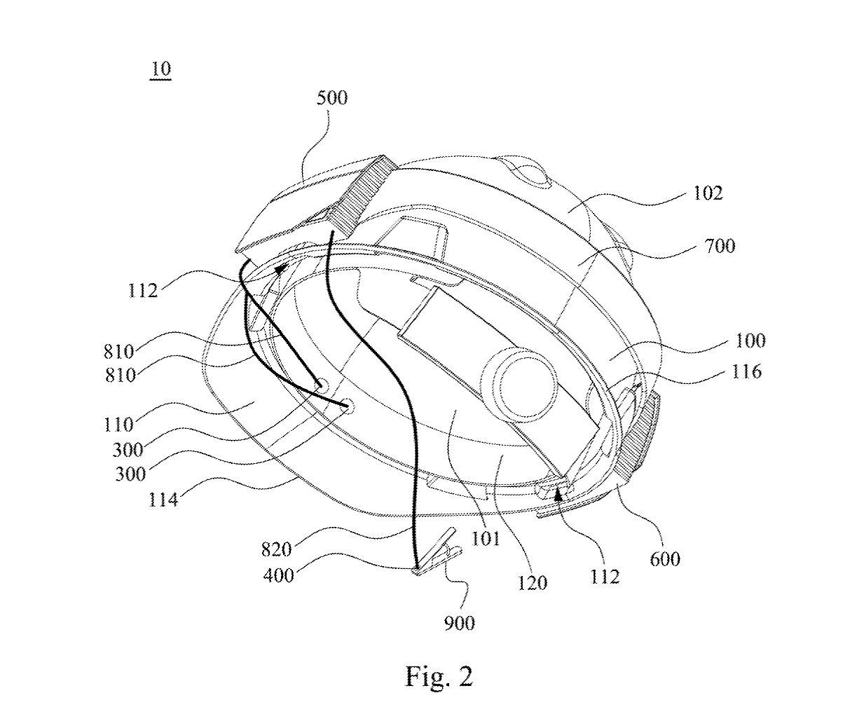

FIG. 2 is a perspective view of the helmet 10 in FIG. 1. As shown in FIG. 2, the helmet 10 includes a cap, such as a safety helmet 100. The fatigue monitoring apparatus can be attached to the safety helmet 100, and includes at least one first electrode 300, at least one second electrode 400, a fatigue computation device 500 and a power supply 600. The safety helmet 100 has a concave surface 101. The concave surface 101 can cover part of the head 30 (See FIG. 1) of the wearer. The first electrode 300 is disposed on the concave surface 101 of the safety helmet 100 to be in contact with one position of the head 30, thereby obtaining first physiological information from the head 30. The second electrode 400 is disposed out of the safety helmet 100 to be in contact with another position of the head 30, thereby obtaining second physiological information. The fatigue computation device 500 is disposed on one side of the safety helmet 100. The fatigue computation device 500 is electrically connected to the first electrode 300 and the second electrode 400, thereby receiving the first physiological information and the second physiological information respectively from the first electrode 300 and the second electrode 400. The fatigue computation device can obtain fatigue information based on the first physiological information and the second physiological information. The fatigue computation device 500 is electrically connected to the power supply 600. The power supply 600 is disposed on another side of the safety helmet 100 opposite to the fatigue computation device 500. In other words, the safety helmet 100 is positioned between the fatigue computation device 500 and the power supply 600.

By the foregoing configuration of the fatigue computation device 500 and the power supply 600, the weight of the helmet 10 can be balanced, and the worker will not feel uncomfortable due to imbalance when wearing the helmet 10, so that the worker can work easily with the helmet 10.

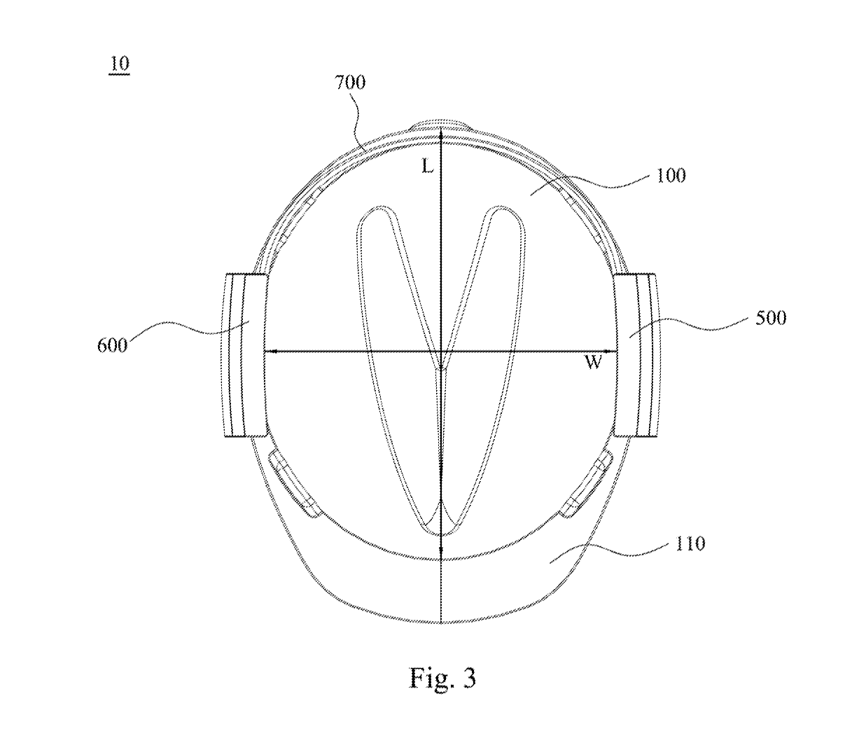

FIG. 3 is a top view of the helmet 10 in FIG. 2. As shown in FIG. 3, in some embodiments of the present invention, the fatigue computation device 500 and the power supply 600 are respectively positioned at two exactly symmetric positions of the safety helmet 100, so as to further balance the mass of the helmet 10 transversely and longitudinally, such that the worker can work more easily when wearing the helmet 10.

In some embodiments, as shown in FIG. 3, the fatigue computation device 500 and the power supply 600 are respectively positioned on the left and the right sides of the safety helmet 100. In other words, when the worker wears the helmet 10, the fatigue computation device 500 is positioned above one ear, and the power supply 600 is positioned above another ear. In particular, the safety helmet 100 has a length L and a width W less than the length L. The direction of the length L is substantially perpendicular to the direction of the width W. The direction of the width W crosses the fatigue computation device 500 and the power supply 600. When the helmet 10 is put on the head 30 (See FIG. 1), the direction of the length L crosses the forehead 31 (See FIG. 1) and the hindbrain 33 (See FIG. 1) of the head 30, and the direction of the width W crosses the ears 32 (See FIG. 1). Because the direction of the width W also crosses the fatigue computation device 500 and the power supply 600, when the helmet 10 is put on the head 30, the fatigue computation device 500 and the power supply 600 can be respectively positioned above the ears 32.

In some embodiments, as shown in FIG. 2, the helmet 10 includes a visor portion 110. The safety helmet 100 has an outer convex surface 102 opposite to the inner concave surface 101. The visor portion 110 is extended from the convex surface 102 of the helmet 10. The visor portion 110 includes a front visor 114 and a rear visor 116 opposite to the front visor 114. When the helmet 10 is put on the head 30 (See FIG. 1), the front visor 114 is positioned above the eyes, so as to protect the eyes from sunlight. In some embodiments, to the surface area of the front visor 114 is greater than which of the rear visor 116, so as to shade the sunlight.

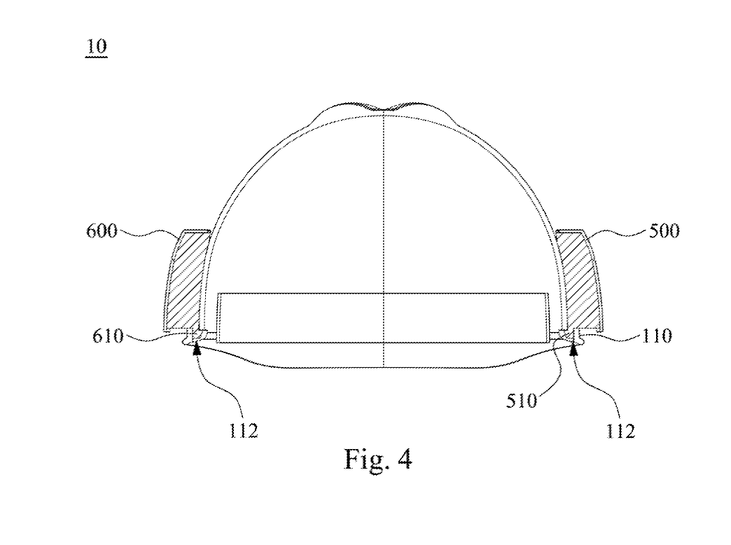

FIG. 4 is a cross-sectional view of the helmet 10 taken along the direction of the width W in FIG. 3. As shown in FIG. 4, the visor portion 110 has a plurality of securing holes 112. The fatigue computation device 500 includes a securing hook 510. The power supply 600 also includes a securing hook 610. The securing hooks 510 and 610 are respectively secured in the securing holes 112 of the visor portion 110, such that the fatigue computation device 500 and the power supply 600 can be fixed on the visor portion 110. Further, in some embodiments, the securing hooks 510 and 610 are removably engaged with the securing holes 112. In other words, the worker can pull the securing hook 510 to make the hook 510 to escape from the securing hole 112, so that the fatigue computation device 500 can be removed from the visor portion 110. Similarly, the worker can also pull the securing hook 610, so that the power supply 600 can be removed from the visor portion 110. In other embodiments of the present invention, the securing means to secure the fatigue computation device 500 and the power supply 600 to the helmet 10 are not limited to the foregoing embodiments, and instead, the fatigue computation device 500 and the power supply 600 can also be secured to the helmet 10 by being adhered or tied or other ways that doesn't damage the helmet, so as to prevent from affecting the protective function of the helmet 10.

In some embodiments, as shown in FIG. 2, the helmet 10 includes a connection member 700. The connection member 700 is electrically connected between the fatigue computation device and the power supply 600. Further, the connection member 700 is positioned on the convex surface 102 of the safety helmet 100. The connection member 700 is curved, and the curvature thereof substantially equals to the curvature of the convex surface 102 of the safety helmet 100, so that the connection member 700 can conformably cover part of the convex surface 102 of the safety helmet 100. The connection member 700 includes a conductive wire therein, so that the power supply 600 can provide electric power to the fatigue computation device 500.

In some embodiments, as shown in FIG. 2, the first electrode 300 is positioned on the inner concave surface 101 of the safety helmet 100 that is close to the front visor 114. Therefore, when the helmet 10 is put on the head 30 (See FIG. 1), the first electrode 300 can be in contact with the forehead 31 (See FIG. 1) of the head 30. The helmet 10 includes at least one wire 810 electrically connected between the first electrode 300 and the fatigue computation device 500. In some embodiments, the helmet 10 includes a wire 820 and a clip 900. The clip 900 is disposed on one end of the wire 820, and another end of the wire 820 is disposed on the fatigue computation device 500. The wire 820 is electrically connected between the second electrode 400 on the clip 900 and the fatigue computation device 500. The clip 900 can clip on any place on the head 30 of the helmet wearer. In an embodiment, the clip 900 is used to clip on the ear 32 (See FIG. 1). By aforementioned configuration, the distance between the second electrode 400 and the first electrode 300 is long enough to differentiate the signals received from the contact positions of the first electrode 300 and second electrode 400.

In some embodiments, as shown in FIG. 2, the helmet 10 includes a liner 120. The liner 120 is disposed on the inner concave surface 101 of the safety helmet 100, and the first electrode 300 is positioned on the liner. The material of the liner 120 is flexible. When the worker wears the helmet 10, the liner 120 can deform corresponding to the shape of the forehead 31, so that the liner 120 can tightly contact the forehead 31, thereby facilitating the first electrode 300 to contact the forehead 31.

When the fatigue monitoring apparatus is in operation, the first electrode 300 can detect the physiological information around the forehead 31 (referred as the first physiological information hereinafter), and the second electrode 400 can detect the physiological information around the ear 32 (referred as the second physiological information hereinafter). Because the forehead 31 is closer to the brain than the ear 32, the first physiological information that the first electrode 300 detects includes the brainwave information, such as the electroencephalogram (EEG), and other physiological noise signal, such as the pulse. Further, because the ear 32 is farer from the brain, the second physiological information that the second electrode 400 detects almost includes only the physiological noise signal. Therefore, the fatigue computation device 500 can obtain the brainwave information based on the difference between the first physiological information and the second physiological information, thereby obtaining the fatigue information.

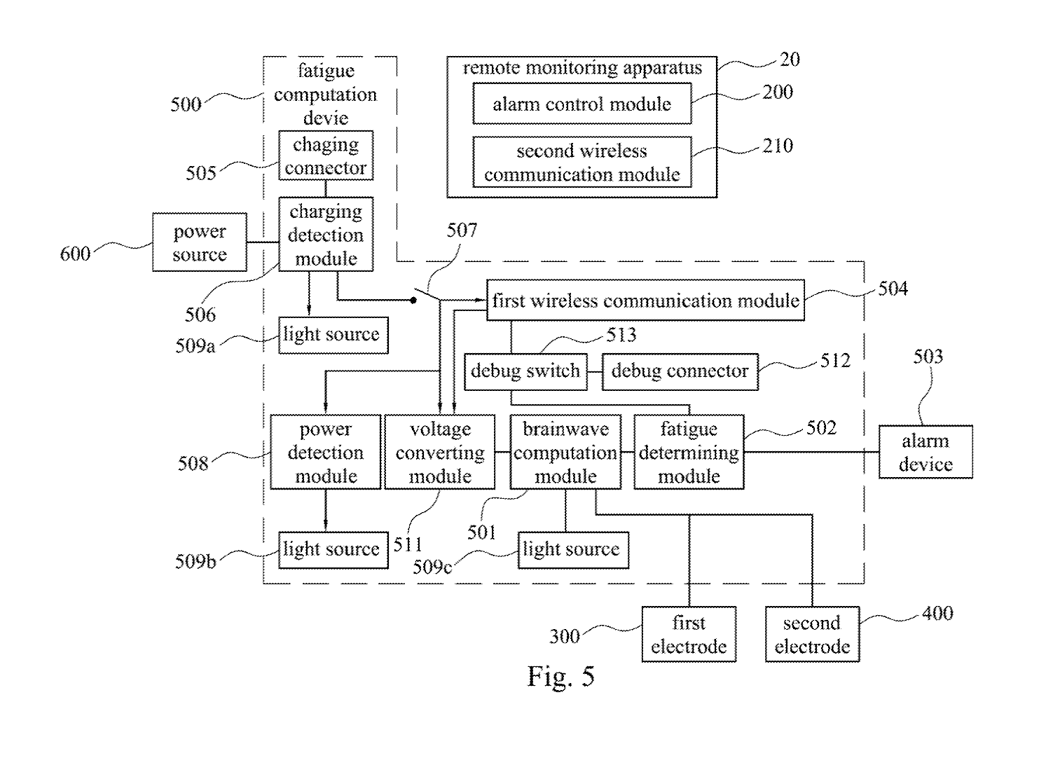

In particular, referring to FIG. 5, this figure is a functional diagram of the safety monitoring system in accordance with one embodiment of the present invention. As shown in FIG. 5, the fatigue computation device 500 includes a brainwave computation module 501 and a fatigue determining module 502. The fatigue determining module 502 is electrically connected to the brainwave computation module 501. The brainwave computation module 501 is electrically connected to the first electrode 300 and the second electrode 400 for obtaining the brainwave information based on the first physiological information and the second physiological information. For example, the brainwave computation module 501 can respectively receive the first physiological information and the second physiological information from the first electrode 300 and the second electrode 400. Then, the brainwave computation module 501 can obtain the difference between the first physiological information and the second physiological information to remove the physiological noise signal, such as the heartbeat or pulse, thereby obtaining the brainwave information. In some embodiments, the brainwave computation module 501 can be implemented by, but is not limited to be implemented by, a brainwave analysis chip.

The fatigue determining module 502 is electrically connected to the brainwave computation module 501 for obtaining the fatigue information based on the brainwave information. For example, when the brainwave information is the EEG, the fatigue determining module 502 can determine the fatigue level of the worker based on the distribution of the EEG's frequency bands. In particular, the EEG mainly includes gamma wave, the beta wave, the alpha wave, the theta wave and the delta wave. The gamma wave refers the EEG with the frequency in the range between 30 Hz and 60 Hz. The beta wave refers to the EEG with the frequency in the range between 18 Hz and 21 Hz. The alpha wave refers to the EEG with the frequency in the range between 9 Hz and 11 Hz. The theta wave refers to the EEG with the frequency in the range between 4 Hz and 7 Hz. The delta wave refers to the EEG with the frequency in the range between 0.5 Hz and 2 Hz.

When the beta wave of the EEG obtained by the fatigue determining module 502 is low, the fatigue determining module 502 can determine the concentration of the worker is low, thereby generating the corresponding fatigue information. When the alpha wave of the EEG obtained by the fatigue determining module 502 is high, the fatigue determining module 502 can determine the worker is tired, thereby generating the corresponding fatigue information. When the theta wave of the EEG obtained by the fatigue determining module 502 is the main wave, the fatigue determining module 502 can determine the worker is sleepy, thereby generating the corresponding fatigue information. Therefore, as long as the fatigue determining module 502 detects one of the three foregoing conditions occurred in the brainwave information, it can generate a corresponding fatigue information. In some embodiments, the fatigue determining module 502 can be implemented by, but is not limited to be implemented by, a micro computer installed with a fatigue evaluation algorithm. Further, in some embodiments, the brainwave computation module 501 and the fatigue determining module 502 electrically connected to each other are not limited to determine the fatigue level, and they can also determine the consciousness such as the mind concentration state or the emotion state. Any physiological state that can be determined based on the brainwave information can be detected and monitored by the safety monitoring system of the present invention, and the monitoring staff only needs to install the required algorithm the foregoing microcomputer. The algorithm corresponding to different brainwave state is well known in the art, and will not be described redundantly.

In some embodiments, as shown in FIG. 5, the helmet 10 (See FIG. 2) includes an alarm device 503. The alarm device 503 is disposed in the fatigue computation device 500 for providing an alarm signal based on the fatigue information. In particular, the alarm device 503 can be a speaker electrically connected to the fatigue determining module 502, so as to make sounds when the fatigue determining module 502 determine that the fatigue of the worker reaches a certain level. Therefore, the alarm device 503 can alert the worker and thereby prevent the worker from keeping working when he is tired. In some embodiments, the alert device 503 can also be other device having alerting function, such as a device generating visual signals, forces, electric currents or any means which can alert the worker. In other words, the alerting means is not limited to the sound.

In some embodiments, as shown in FIG. 5, the helmet 10 (See FIG. 2) includes a first wireless communication module 504. The first wireless communication module 504 is disposed in the fatigue computation device 500, and is electrically connected to the fatigue determining module 502. The remote monitoring apparatus 20 includes a second wireless communication module 210. The first wireless communication module 504 is in wireless communication with the second wireless communication module 210. Therefore, the first wireless communication module 504 can wirelessly transmit the fatigue information obtained by the fatigue determining module 502 to the second wireless communication module 210 of the remote monitoring apparatus 20. As such, the user of the remote monitoring apparatus 20 can monitor the physiological state of the worker wearing the helmet 10 (See FIG. 2), so as to immediately know that whether the worker is tired. In some embodiments, the first wireless communication module 504 and the second wireless communication module 210 comply with the GPRS protocol. However, the present invention is not limited to this. Any available wireless communication protocol can be utilized in the first and second wireless communication modules.

In some embodiments, as shown in FIG. 5, the remote monitoring apparatus 20 includes an alarm control module 200. The alarm control module 200 is electrically connected to the second wireless communication module 110. The alarm device 503 is electrically connected to the first wireless communication module 504. The alarm control module 200 can generate a control signal. The alarm device 503 can generate alarm based on the control signal. For example, when the user of the remote monitoring apparatus 20 perceives that the worker wearing the helmet 10 (See FIG. 2) is tired, he can utilize the alarm control module 200 to generate the control signal. The second wireless communication module 210 can wirelessly transmit the control signal to the first wireless communication module 504 in the helmet 10, and the first wireless communication module 504 can transmit the control signal to the alarm device 503, so as the make the alarm device 503 generate alarm, such as voice message, to remind the worker to take a break. In some embodiments, the remote monitoring device 20 doesn't include the alarm control module 200, and instead, the remote monitoring device 20 records the fatigue information of the workers from every fatigue computation device 500. Further, the remote monitoring apparatus 20 can transmit relating parameters, such as the threshold of EEG's frequency bands, masks of noises, or etc., to the fatigue determining module 520, so as to adjust the computation process for determining fatigue or result.

In some embodiments, as shown in FIG. 5, the helmet 10 (See FIG. 2) includes a charging connector 505, a charging detection module 506 and a light source 509a. The charging connector 505 is electrically connected to the power supply 600. The charging detection module 506 is also electrically connected the power supply 600. The charging detection module 506 detects the power status of the power supply 600 and generates a power status signal of the power supply 600. The light source 509a is electrically connected to the charging detection module 506 for emitting a light based on the received power status signal. In particular, the charging connector 505 can be an electrical power connector, such as an USB connector. The charging connector 505 can be plugged by an external power source, and can receive the power from the external power source, and then transmit the power to charge the power supply 600. When the power status of the power supply is charged enough as a full power state, the charging detection module 506 can control the light source 509a to emit a light, thereby reminding the helmet holder that the charging process is finished. The charging detection module 506 can be implemented by, but is not limited to be implemented by, an IC capable of detecting the power status. The power supply 600 can be, but is not limited to be, a rechargeable battery.

In some embodiments, as shown in FIG. 5, the helmet 10 (See FIG. 2) includes a power detection module 508 and a light source 509b. The power detection module 508 is electrically connected to the power supply 600 for detecting the power status of the power supply 600. When the power status of the power supply 600 is low, the power detection module 508 can control the light source 509b to emit a light, so as to remind the helmet holder that the power is low. The power detection module 508 can generates a low power signal when the power status of the power supply 600 is low. The light source 509b is electrically connected to the power detection module 508 for emitting a light when receiving the low power signal. The power detection module 508 can be implemented by, but is not limited to be implemented by, an IC capable of detecting the power status. Further, the helmet 10 includes a light source 509c electrically connected to the brainwave computation module 501 or the fatigue determining module 502. The light source 509c emits a light when the brainwave module 501 or the fatigue determining module 502 is in operation, so as to inform the worker that the fatigue computation device 500 can normally work. In some embodiments, the light sources 509a, 509b and 509c emit lights in different colors, so as to facilitate the worker or helmet holder to distinguish the lights emitted by the light sources 509a, 509b and 509c. For example, the lights emitted by the light source 509a, the light source 509b and the light source 509c can be, but are not limited to be, orange, red and green. In some embodiments, the light source 509a, the light source 509b and the light source 509c can be, but are not limited to be, Light-Emitting Diodes (LEDs) connecting to light guide components.

In some embodiments, as shown in FIG. 5, the helmet 10 (See FIG. 2) includes a debug connector 512 and a debug switch 513. The debug connector 512 and the debug switch 513 are both disposed in the fatigue computation device 500. One end of the debug switch 513 is electrically connected to the fatigue determining module 502, and another end of the debug switch 513 is alternatively electrically connected to the debug connector 512 or the first wireless communication module 504. When an external debug device is plugged in the debug connector 512, the debug switch 513 can be switched to connect the debug connector 512 and the fatigue determining module 502. In this situation, the external debug device can execute the debug process of the fatigue computation device 500. When the debug connector 512 is not plugged by any external device, the debug switch 513 can be switched to connect the first wireless communication module 504 and the fatigue determining module 502.

In some embodiments, as shown in FIG. 5, the helmet 10 (See FIG. 2) includes a switch 507 electrically connected to the power supply 600. In particular, the switch 507 is electrically connected among the power supply 600 and the brainwave computation module 501 and the fatigue determining module 502, so as to provide or not provide power to the brainwave computation module 501 and the fatigue determining module 502. In some embodiments, the helmet 10 includes a voltage converting module 511 electrically connected between the brainwave computation module 501 and the power supply 600, so as to convert the power from the power supply 600 to an appropriate voltage level, thereby preventing the brainwave computation module 501 and the fatigue determining module 502 from damaged due to the high voltage.

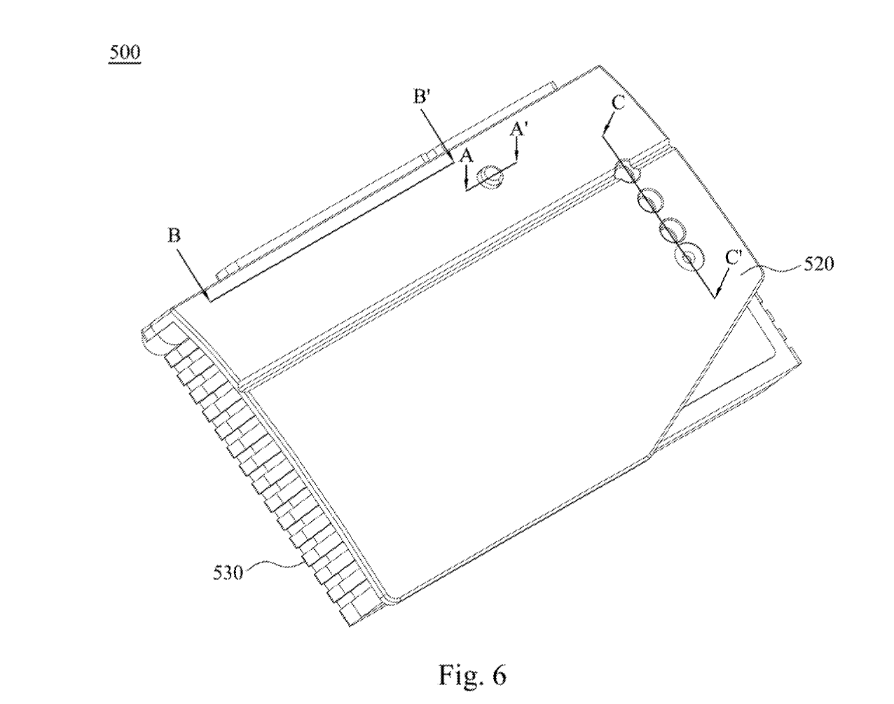

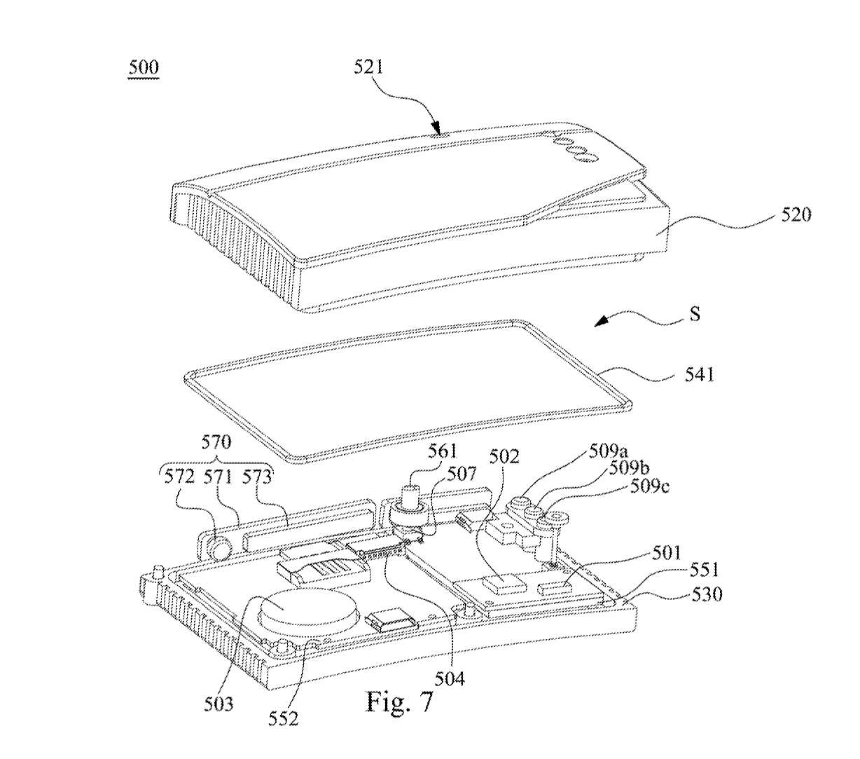

FIG. 6 is a perspective view of the fatigue computation device 500 in accordance with one embodiment of the present invention. FIG. 7 is an explosive view of the fatigue computation device 500. As shown in FIG. 7, the fatigue computation device 500 includes an upper cover 520, a lower cover 530 and a first waterproof structure 541. The upper cover 520 and the lower cover 530 define an containing space S therebetween. The first waterproof structure 541 is sandwiched between the upper cover 520 and the lower cover 530, so as to protect the containing space S from water permeating. The material of the first waterproof structure 541 is flexible material, such as the rubber, so as to block the water out of the containing space S.

In some embodiments, as shown in FIG. 7, the fatigue computation device 500 includes two circuit boards 551 and 552. The circuit boards 551 and 552 are both accommodated in the containing space S, and are both disposed on the lower cover 530. The brainwave computation module 501, the fatigue determining module 502, the switch 507 and the light sources 509a, 509b and 509c are all accommodated in the containing space S. In particular, the brainwave computation module 501, the fatigue determining module 502, the switch 507 and the light sources 509a, 509b and 509c are all disposed on the circuit board 551. Further, the alarm device 503 and the first wireless communication module 504 are also accommodated in the containing space S, and are disposed on the circuit board 552.

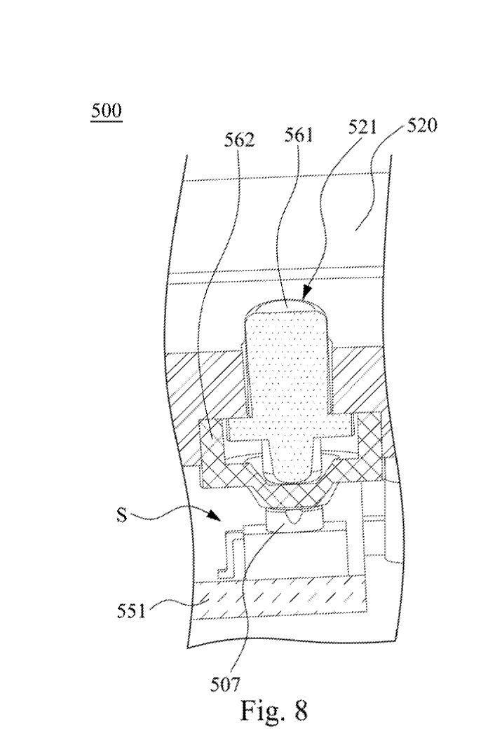

FIG. 8 is a cross-sectional view of the fatigue computation device 500 taken along A-A′ line in FIG. 6. As shown in FIG. 8, the fatigue computation device 500 includes a pressing structure 561, such as a button or pad. The upper cover 520 has a through hole 521. The pressing structure 561 is movably coupled to the through hole 521. The projection position that the pressing structure 561 is perpendicularly projected to the circuit board 551 partially overlaps with the switch 507. In other words, the pressing structure 561 is positioned through the through hole 521, and is movable in the through hole 521. The switch 507 is positioned under the pressing structure 561. When the pressing structure 561 is pressed and moves downwardly, it presses the switch 507, so as to enable or disable the fatigue computation device 500. Part of pressing structure 561 is preferably extended out of the through hole 521 of the upper cover 520, so that the worker can touch and operate the pressing structure 561.

In some embodiments, as shown in FIG. 8, the fatigue computation device 500 includes a flexible member 562. The flexible member 562 is sandwiched between the pressing structure 561 and the switch 507. When the pressing structure 561 is pressed and moves downwardly, it can press the flexible member 562 to press the switch 507. When the pressing structure 561 stops being pressed, the flexible member 562 bounces back and thereby makes the pressing structure 561 move back to the original position. In some embodiments, the flexible member 562 covers part of the pressing structure 561 in the containing space S, and is tightly fitted with the upper cover 520, so as to prevent the water from getting into the containing space S through the through hole 521.

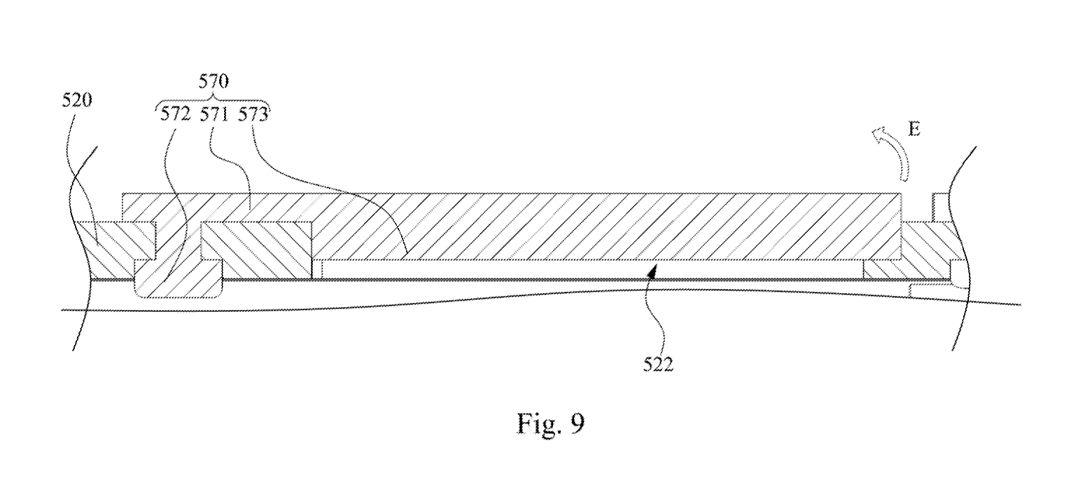

FIG. 9 is a cross-sectional view of the fatigue computation device 500 taken along B-B′ line in FIG. 6. As shown in FIG. 9, in some embodiments, the fatigue computation device 500 includes a sealing cap 570, and the upper cover 520 has at least one opening 522. The sealing cap 570 can seals the opening 522, and can also be pulled out of the opening 522, so that the pluggable electrical component, such as the subscriber identity module (SIM) card or the memory card, can be plugged into the opening 522 of the fatigue computation device 500. In particular, the sealing cap 570 includes a flexible body 571, a fixing part 572 and a waterproof part 573. The fixing part 572 is fixed on the upper cover 520. The waterproof part 573 is disposed on the flexible body 571. The flexible body 571 is bendable relative to the fixing part 572. For example, the flexible body 571 can be bended along the direction of the arrow E, so that the waterproof part 573 can be moved out of the opening 522. When the flexible body 571 is not bended, the waterproof part 573 is sealed with the opening 522, so as to protect the fatigue computation device 500 from water permeating through the opening 522. The material of the waterproof part 573 is preferably the flexible material, such as the rubber, so as to block the water.

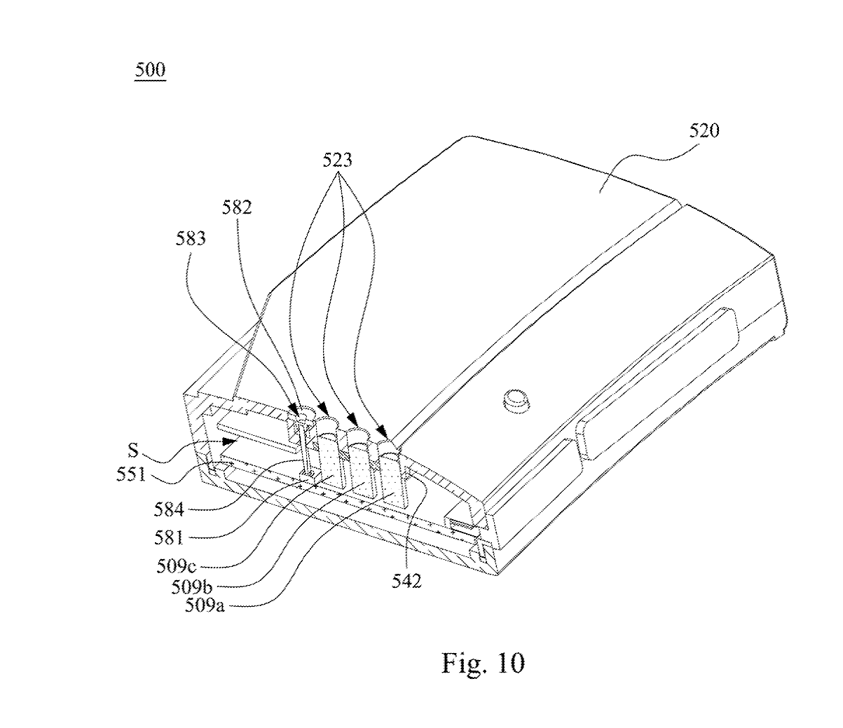

FIG. 10 is a cross-sectional view of the fatigue computation device 500 taken along C-C′ line in FIG. 6. As shown in FIG. 10, in some embodiments, the light sources 509a, 509b and 509c are all partially positioned in the containing space S, and are partially exposed on the upper cover 520. In particular, the upper cover 520 has a plurality of light outgoing holes 523. The light outgoing holes 523 are respectively positioned above the light sources 509a, 509b and 509c, so as to expose the light sources 509a, 509b and 509c. The fatigue computation device 500 includes a second waterproof structure 542. The second waterproof structure 542 enfolds the light sources 509a, 509b and 509c, and can be sealed with the upper cover 520, so as to protect the containing space S from water permeating through the light outgoing hole 523.

In some embodiments, as shown in FIG. 10, the fatigue computation device 500 includes a reset switch 581 and a rest button 582. The reset switch 581 is disposed on the circuit board 551. The reset switch 581 can reset the brainwave computation module 501 (See FIG. 7) and the fatigue determining module 502 (See FIG. 7) to the original state or the beginning of the fatigue determination process. The reset button 582 is disposed on the upper cover 520, and the projection position that the reset button 582 is perpendicularly projected to the circuit board 551 at least partially overlaps with the reset switch 581. The reset button 582 includes a pressing part 583. The pressing part 583 is concave toward the reset switch 581 inwardly in order to prevent false triggering. The pressing part 583 is flexible. An auxiliary member 584 is positioned between the pressing part 583 and the reset button 582. The worker can utilize a thin object, such as the pointed tip of the pen, to press the pressing part 583. When the pressing part 583 is pressed and moves downwardly, it can push the auxiliary member 584, thereby pressing the reset button 582. In some embodiments, the pressing part 583 can directly contact with the reset button 582 without auxiliary member 584 intervening therebetween.

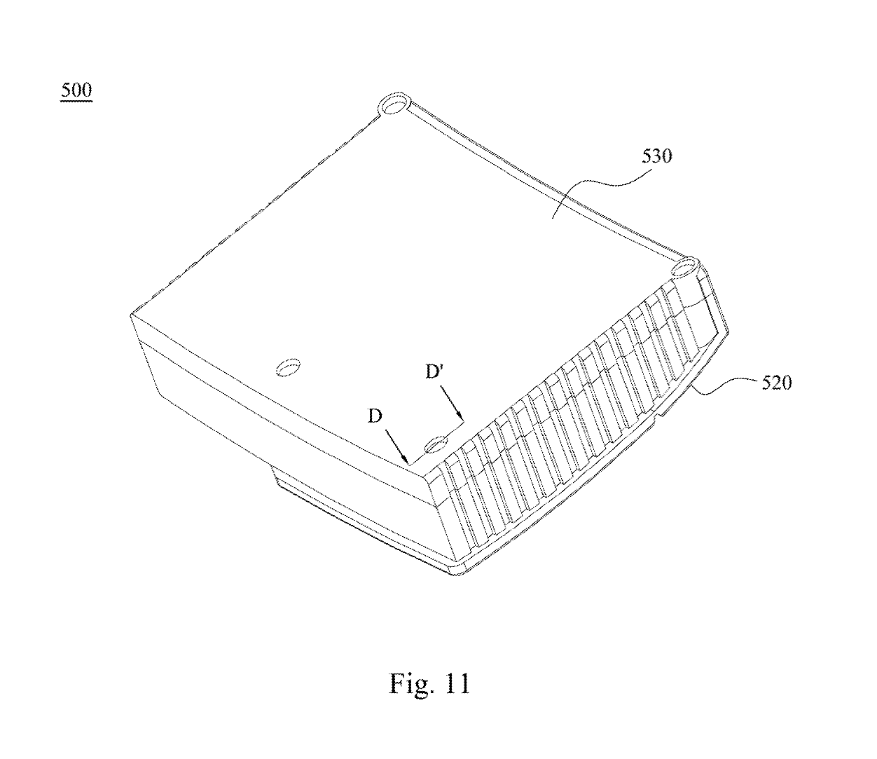

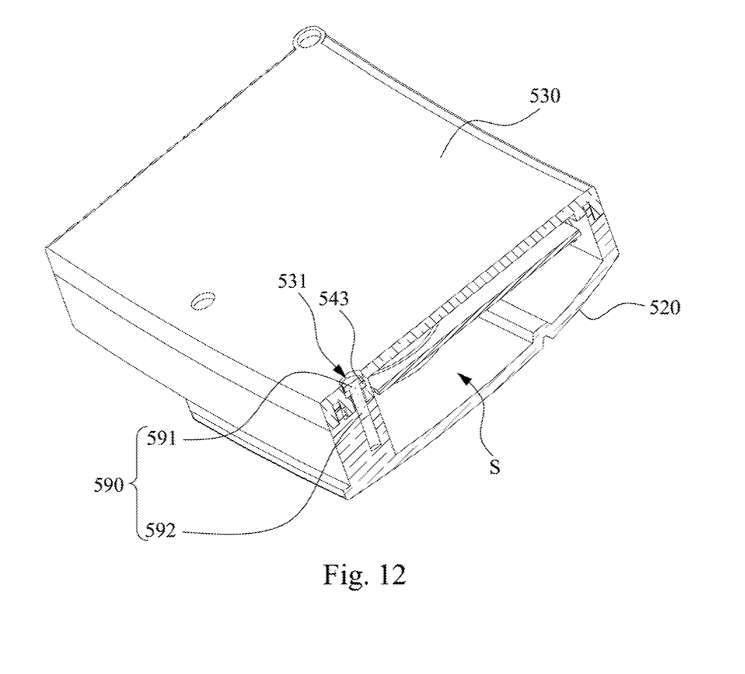

FIG. 11 is another perspective view of the fatigue computation device 500 in accordance with one embodiment of the present invention. FIG. 12 is a cross-sectional view of the fatigue computation device 500 taken along D-D′ line in FIG. 11. As shown in FIG. 12, the fatigue computation device 500 includes a fastener 590 and a third waterproof structure 543. The fastener 591 can fasten the upper cover and the lower cover 530. The third waterproof structure 543 enfolds the fastener 590. For example, the fastener 590 can be a screw which includes a screw head 591 and a screw leg 592 connected to the screw head 591. The lower cover 530 has a screw hole 531. The screw head 591 is screwed into the screw hole 531 of the lower cover 530. The third waterproof structure 543 can enfold the screw leg 592 in the screw hole 531, and can be tightly fitted with the wall of the screw hole 531, so as to protect the containing space S from water permeating through the screw hole 531. In some embodiments, the positions, amounts, directions of the elements on the cover (including the upper cover 520 and the lower cover 530) can be varied according to the necessity, and the positions, amounts, directions of the circuit elements within the cover can also be varied, and are not limited to the foregoing embodiments.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A fatigue detecting helmet comprising:
a cap having a concave inner surface;
at least one first electrode disposed on the concave inner surface of the cap to be in contact with one position on a head of a wearer, thereby obtaining a first physiological information of the wearer;
at least one second electrode disposed out of the cap to be in contact with another position on the head of the wearer, thereby obtaining a second physiological information of the wearer;
a fatigue computation device disposed on one side of the cap, the fatigue computation device comprising a brainwave computation module and a fatigue determining module, wherein the brainwave computation module is electrically connected to the first electrode and the second electrode for obtaining a brainwave information based on the first physiological information and the 15 second physiological information, wherein the fatigue determining module is electrically connected to the brainwave computation module for obtaining a fatigue information of the wearer based on the brainwave information; and
a power supply disposed on another side of the cap opposite to the fatigue computation device, wherein the fatigue computation device is electrically connected to the power supply.

2. The fatigue detecting helmet of claim 1, wherein the fatigue computation device and the power supply are respectively positioned on two symmetric positions of the cap.

3. The fatigue detecting helmet of claim 1, wherein the cap comprises:
a visor portion, wherein the cap has a convex outer surface, and the visor portion is disposed on the outer convex surface of the cap, wherein the visor portion has a plurality of securing holes and the fatigue computation device and the power supply both comprises at least one securing hook, and the securing hooks are respectively secured in the securing holes of the visor portion.

4. The fatigue detecting helmet of claim 1, wherein the fatigue computation device comprises:
an upper cover;
a first waterproof structure;
a lower cover, wherein the first waterproof structure is sandwiched between the upper cover and the lower cover, wherein the upper cover and the lower cover define a containing space therebetween, and the brainwave computation module and the fatigue determining module are accommodated in the containing space;
a circuit board accommodated in the containing space, wherein the brainwave computation module and the fatigue determining module are disposed on the circuit board;
a switch disposed on the circuit board, wherein the switch is electrically connected to the power supply; and
a pressing structure, wherein the upper cover has a through hole, wherein the pressing structure is movably coupled to the through hole, wherein a projection position that the pressing structure is perpendicularly projected to the circuit board partially overlaps with the switch.

5. The fatigue detecting helmet of claim 1, further comprising:
a power detection module electrically connected to the power supply for detecting a power of the power supply and generating a low power signal when the power of the power supply is low;
a charging connector electrically connected to the power supply;
a charging detection module electrically connected the power supply for detecting the power of the power supply and generating a full power signal when the power of the power supply is full; and
a light source electrically connected to the power detection module and the charging detection module for emitting a light when receiving the low power signal or the full power signal.

6. The fatigue detecting helmet of claim 1, further comprising:
a first wireless communication module disposed in the fatigue computation device for wirelessly transmitting the fatigue information to a remote monitoring apparatus.

7. The fatigue detecting helmet of claim 1, further comprising:
an alarm device disposed in the fatigue computation device for providing an alarm signal based on the fatigue information.

8. A fatigue monitoring apparatus capable of being attached to a cap, the fatigue monitoring apparatus comprising:
at least one first electrode for being in contact with a first position of a head, thereby obtaining a first physiological information;
at least one second electrode for being in contact with a second position of the head, thereby obtaining a second physiological information, wherein the second position is farer from a brain of the head than the first position;
a fatigue computation device capable of being disposed on one side of the cap, the fatigue computation device comprising a brainwave computation module and a fatigue determining module, wherein the brainwave computation module is electrically connected to the first electrode and the second electrode for obtaining a brainwave information based on the first physiological information and the second physiological information, wherein the fatigue determining module is electrically connected to the brainwave computation module for obtaining a fatigue information based on the brainwave information; and
a power supply capable of being disposed on another side of the cap opposite to the fatigue computation device, wherein the fatigue computation device is electrically connected to the power supply.

9. The fatigue monitoring apparatus of claim 8, further comprising:
a first wireless communication module disposed in the fatigue computation device for wirelessly transmitting the fatigue information to a remote monitoring apparatus.

10. The fatigue monitoring apparatus of claim 8, further comprising:
an alarm device disposed in the fatigue computation device for providing an alarm signal based on the fatigue information.

11. The fatigue monitoring apparatus of claim 8, wherein the fatigue computation device and the power supply are connected by a connection member and capable of being respectively positioned on two symmetric positions of the cap.

12. The fatigue monitoring apparatus of claim 8, wherein the fatigue computation device comprises:
an upper cover;
a first waterproof structure;
a lower cover, wherein the first waterproof structure is sandwiched between the upper cover and the lower cover, wherein the upper cover and the lower cover define a containing space therebetween, and the brainwave computation module and the fatigue determining module are accommodated in the containing space;
a circuit board accommodated in the containing space, wherein the brainwave computation module and the fatigue determining module are disposed on the circuit board;
a switch disposed on the circuit board, wherein the switch is electrically connected to the power supply; and
a pressing structure, wherein the upper cover has a through hole, wherein the pressing structure is movably coupled to the through hole, wherein a projection position that the pressing structure is perpendicularly projected to the circuit board partially overlaps with the switch.

13. The fatigue monitoring apparatus of claim 8, further comprising:
a power detection module electrically connected to the power supply for detecting a power of the power supply and generating a low power signal when the power of the power supply is low;
a charging connector electrically connected to the power supply;
a charging detection module electrically connected the power supply for detecting the power of the power supply and generating a full power signal when the power of the power supply is full; and
a light source electrically connected to the power detection module and the charging detection module for emitting a light when receiving the low power signal or the full power signal.

14. A safety monitoring system comprising:
a monitoring server having a first wireless communication module; and
at least one fatigue monitoring apparatus attached to a cap, the fatigue monitoring apparatus comprising at least one first electrode, at least one 20 second electrode, a fatigue computation device and a power supply, wherein the first electrode and the second electrode are respectively in contact with different positions of a head for respectively obtaining a first physiological information and a second physiological information, wherein the fatigue computation device obtains a brainwave information based on the difference between the first physiological information and the second physiological information, and obtains a fatigue information based on the brainwave information, wherein the power supply is electrically connected to the fatigue computation device for providing power, wherein the fatigue computation device and the power supply are disposed on opposite sides of the cap, wherein the fatigue monitoring apparatus comprises a second wireless communication module;
wherein the first wireless communication module is in wireless communication with the second wireless communication module, wherein the monitoring server receives the fatigue information from the fatigue monitoring apparatus.

15. The safety monitoring system of claim 14, wherein the fatigue computation device and the power supply are respectively positioned on two symmetric positions of the cap.

16. The safety monitoring system of claim 14, wherein the monitoring server has an alarm control module and the at least one fatigue monitoring apparatus has an alarm device, the alarm control module generates a control signal, the first wireless communication module transmits the control signal to the second wireless communication module and the second wireless communication module transmits the control signal to the alarm device for providing an alarm signal.

17. The safety monitoring system of claim 14, wherein the monitoring server records the fatigue information from the fatigue monitoring apparatus and transmits parameters to the fatigue computation device, and the fatigue computation device adjust computation according to the parameters.

18. The safety monitoring system of claim 14, wherein the fatigue computation device comprises:
an upper cover;
a first waterproof structure;
a lower cover, wherein the first waterproof structure is sandwiched between the upper cover and the lower cover, wherein the upper cover and the lower cover define a containing space therebetween, and the brainwave computation module and the fatigue determining module are accommodated in the containing space;
a circuit board accommodated in the containing space, wherein the brainwave computation module and the fatigue determining module are disposed on the circuit board;
a switch disposed on the circuit board, wherein the switch is electrically connected to the power supply; and
a pressing structure, wherein the upper cover has a through hole, wherein the pressing structure is movably coupled to the through hole, wherein a projection position that the pressing structure is perpendicularly projected to the circuit board partially overlaps with the switch.

19. The safety monitoring system of claim 14, wherein the at least one fatigue monitoring apparatus further comprises:
a power detection module electrically connected to the power supply for detecting a power of the power supply and generating a low power signal when the power of the power supply is low;
a charging connector electrically connected to the power supply;
a charging detection module electrically connected the power supply for detecting the power of the power supply and generating a full power signal when the power of the power supply is full; and
a light source electrically connected to the power detection module and the charging detection module for emitting a light when receiving the low power signal or the full power signal.

20. The safety monitoring system of claim 14, wherein the at least one fatigue monitoring apparatus is attached to a cap without damaging the structure of the cap.
